(19) United States
(12) Patent Application Publication
(10) Pub. No.: US 2003/0029460 A1
(43) Pub. Date: Feb. 13, 2003

(54) Extruded earplug

(76) Inventor: Jim Tiemens, Laguna Nigel, CA (US)

Correspondence Address: LEON D. ROSEN, FREILICH, HORNBAKER & ROSEN, Suite 1220, 10960 Wilshire Blvd., Los Angeles, CA, 90024, US

(21) Appl. No.: 10/212,288

(22) Filed: Aug. 1, 2002

Related U.S. Application Data
(60) Provisional application No. 60/311,440, filed on Aug. 10, 2001.
(60) Provisional application No. 60/311,441, filed on Aug. 10, 2001.
(60) Provisional application No. 60/311,610, filed on Aug. 10, 2001.

(51) Int. Cl. A61F 11/00

(52) U.S. Cl. 128/864

(57) ABSTRACT

Earplugs produced by extrusion of a foamable polymer through an extrusion head opening (14), are severed and shaped to produce a largely symmetric rounded end (27, 28) and/or compression bands (190, 192, 204) for stiffening. Severing of an earplug at the extrusion head is accomplished by two or more cutting blades (50, 52) that move in largely opposite directions across one another to provide a symmetric cut that foams into a largely symmetric rounded earplug end. Compression bands are formed along the earplug to stiffen it, by two or more compression dies (221-224) spaced about the axis of the earplug. An earplug with a stiffening sleeve (116) that is surrounded by a foam covering (124) and that is filled with a string (122) of the same foam, is severed so at the opposite ends of the earplug the covering ends (160) and string ends (162) are rounded even though the sleeve end (164) is cut perpendicular to the earplug axis (166).

### CROSS-REFERENCE TO RELATED APPLICATION

Applicant claims priority from provisional patent applications 60/311,440, 60/311,441, and 60/311,610, all filed Aug. 10, 2001.

### BACKGROUND OF THE INVENTION

One common type of disposable earplug includes a largely cylindricallyshaped body formed completely of slow recovery foam material. Such earplug is installed by rolling between the fingers to a small diameter and inserting into the ear canal where it expands against the ear canal over a period of perhaps thirty seconds. Such earplugs can be formed by punching a plug out of a plate of slow recovery material, by molding individual earplugs in individual molds, and by extruding material that is cut into earplugs. PCT Publication WO 02/26465 describes the use of a rotary knife cutter to cut an extrusion at the die face of the extruder. Although the foamable material continues to foam and forms a somewhat rounded surface after it is cut at the die face, the end of the earplug tends to form a non-symmetric rounded end. A non-symmetric end, even if rounded, creates an appearance of poor quality. For foam earplugs that are not of the slow recovery type that is rolled into a small diameter, an off center rounding can interfere with insertion of the earplug into the ear canal. It is noted that slow recovery earplugs are convenient to use, but have the disadvantage that they tend to become dirty when rolled between the fingers of workers with dirty hands prior to insertion in the ear canal.

Another type of commonly used earplug is formed of rapid recovery resilient foam material. Such an earplug can be pressed into the ear canal without rolling to a smaller diameter, but is difficult to install without means for stiffening it against column-like collapse. One approach to stiffening the earplug is to provide a core that extends along the axis of the earplug, the core being constructed of material stiffer than that of the covering that surrounds the core and that is compressed by the ear canal. U.S. Pat. No. 5,753,015 describes the feeding of a small diameter rod-shaped core through an extrusion head while resilient foam material is extruded around the core. The resulting extrusion is cut into individual earplugs by a knife blade. Other means for cutting include hot wire, water jet, and laser. Although this patent describes means for forming the earplugs, as to round their ends while the foam has not yet solidified, this is difficult to do without hardening the foam so it loses its resilience at its compressed ends.

Extruded earplugs and methods and equipment for forming them, which provided largely symmetric earplugs that could be easily inserted into the ear canal would be value.

### SUMMARY OF THE INVENTION

In accordance with one embodiment of the present invention, an extruded earplug apparatus is provided, which has symmetrically formed portions such as an end that is inserted into the ear canal, and which is easily constructed. In a process wherein an extrusion is cut into individual earplugs as it emerges from the extrusion head, the cutting of the extrusion is accomplished by at least two cutting blades that move in largely opposite directions across the extrusion. This produces largely symmetric deformations of the end of an earplug lying adjacent to the extrusion head, so the additional foaming and rounding of the earplug end is largely symmetric to produce an earplug of quality appearance and easy insertability.

One type of extruded earplug includes a sleeve-shaped core and rapid recovery resilient foam material covering the sleeve and filling the inside of the sleeve. At an end of the extrusion head, the sleeve is cut perpendicular to the earplug axis, but the covering and string-like foam within the sleeve both become rounded. This avoids the appearance of a projecting core which might scrape against the ear canal and create discomfort during installation.

An earplug formed solely of rapid recovery resilient foam is stiffened by one or more bands where the foam is compressed, the bands extending in planes perpendicular to the axis of the earplug. The bands are formed by a plurality of compressing dies that symmetrically compress the foam at locations near the extrusion head.

The novel features of the invention are set forth with particularity in the appended claims. The invention will be best understood from the following description when read in conjunction with the accompanying drawings.

### BRIEF DESCRIPTION OF THE DRAWINGS

FIG. 1 is a partially sectional side view of an extrusion head and of an extrusion being cut to form an earplug.

FIG. 2 is a side elevation view of the final earplug created by the process of FIG. 1.

FIG. 3 is a view taken on line 3-3 of FIG. 1, showing only the cutting blades and actuators in their closed severing positions.

FIG. 3A is a view of the apparatus of FIG. 3, with the cutting blades in their open position.

FIG. 4 is a front elevation view of an extrusion shearing apparatus of another embodiment of the invention, in a first stationary position.

FIG. 4A is a view similar to FIG. 4, with the apparatus in a second stationary position.

FIG. 5 is an isometric view of an earplug resulting from severing an extrusion by a single knife blade.

FIG. 6 is a partial isometric view of a pair of cutting blades that rotate in opposite directions in accordance with another embodiment of the invention.

FIG. 7 is a partially sectional side elevation view of an extrusion head, a compressing apparatus, and a chain of earplugs resulting from the extrusion and compressing process.

FIG. 7A is a side elevation view of one of the earplugs of the chain of FIG. 7 after it has been severed from the chain.

FIG. 8 is a partial sectional side view of an extrusion head and compressing apparatus of another embodiment of the invention, in a process where a sleeve-shaped core is passed through the center of the extrusion head, the figure also showing a portion of a chain of earplugs resulting from the process.

FIG. 9 is a partially sectional side view of one of the earplugs of the chain of earplugs of FIG. 8, after it has been severed from the rest of the chain.

FIG. 10 is a sectional view of an extrusion head similar to that of FIG. 8, but with cutting blades for cutting individual earplugs out of the extrusion at a location adjacent to the extrusion head.

FIG. 11 is a sectional view of one of the complete earplugs created by the process of FIG. 10.

FIG. 12 is a sectional view taken on line 12-12 of FIG. 11.

FIG. 13 is a partially sectional view of an extrusion head and a short length of extrusion that forms an earplug, and showing cutting blades and compression dies applied to the extrusion.

FIG. 14 is a sectional view of the earplug resulting from the process of FIG. 13.

FIG. 15 is a view taken on line 15-15 of FIG. 13, showing a set of four compression dies in their open position.

FIG. 16 is a view of a set of three compression dies in their open position.

FIG. 17 is a sectional view of an earplug and compression dies applied thereto, of another embodiment of the invention.

FIG. 18 is a sectional view of only the earplug of FIG. 17, taken on line 18-18 thereof.

FIG. 19 is a partially sectional view of an extrusion head, cutting blades in, an open position, and compression dies in an open position, for forming an earplug of another embodiment of the invention.

FIG. 20 is a sectional view of an earplug constructed in accordance with the process of FIG. 19.

FIG. 21 is an elevation view of apparatus useful in the production of earplugs that are severed at the extrusion head.

FIG. 22 is a sectional side view of an earplug of another embodiment of the invention.

FIG. 23 is a side elevation view of an earplug of another embodiment of the invention.

### DESCRIPTION OF THE PREFERRED EMBODIMENTS

FIG. 1 illustrates an extrusion head 10 with an extrusion die 12 having a substantially round opening 14, and a quantity of flowable foam polymer material 16 that is pressurized to emerge in a forward direction F from the extrusion head. As the material emerges, it rapidly foams, and in so doing it grows in diameter to a final diameter A that is perhaps two to five times the diameter of the round extrusion die opening 14. The foaming of the polymer material is indicated by the appearance of and increased density of bubbles representing the foam. In the process of FIG. 1, the extrusion is cut into earplugs each having a length of about 30 mm (10 mm to 50 mm). It is highly desirable to severe the extrusion 22 that projects by at least 1 cm forward of the extrusion head, from material emerging from the extrusion head, close to the plane 24 at the front end of the extrusion head. At the severing location 26, the foamable material has not completely foamed, and when cut there the cut surface will continue to foam and become rounded. A rounded earplug end is desirable, because it results in a tapering that facilitates insertion of the earplug into the ear canal.

One way to severe the extrusion 22 from the foamable material in the extrusion head, is with a knife blade that moves rapidly across the axis 30 of the extrusion at the location 26. While a knife blade that rapidly moves across the location 26 will reliably sever the extrusion from the material at the extrusion head opening, this can result in a non-symmetric earplug end. FIG. shows an example where a blade has severed an earplug end, resulting in one side 40 of the earplug end projecting more forward of an opposite side 42. Such non-symmetry creates the impression of poor quality. Also, a projection at 40 at one side can make it somewhat more difficult to insert the earplug into the ear canal.

In accordance with one aspect of the present invention, applicant severs the extrusion 22 of FIG. 1 from the material emerging from the extrusion head opening 14, by a plurality of cutting or shearing blades. FIG. 3A shows the cutting blades 50,52 in an open position, wherein center parts 54, 56 of the cutting edges 60, 62 are separated to allow the extrusion to pass between the blades. FIG. 3 shows the cutting blades 50, 52 in their closed position, wherein they have moved across one another to shear the foamable material which has only started to foam. A pair of actuators 64, 66 move the cutting blades toward and away from each other. The movement is rapid compared to the rate of extrusion of material out of the extrusion head.

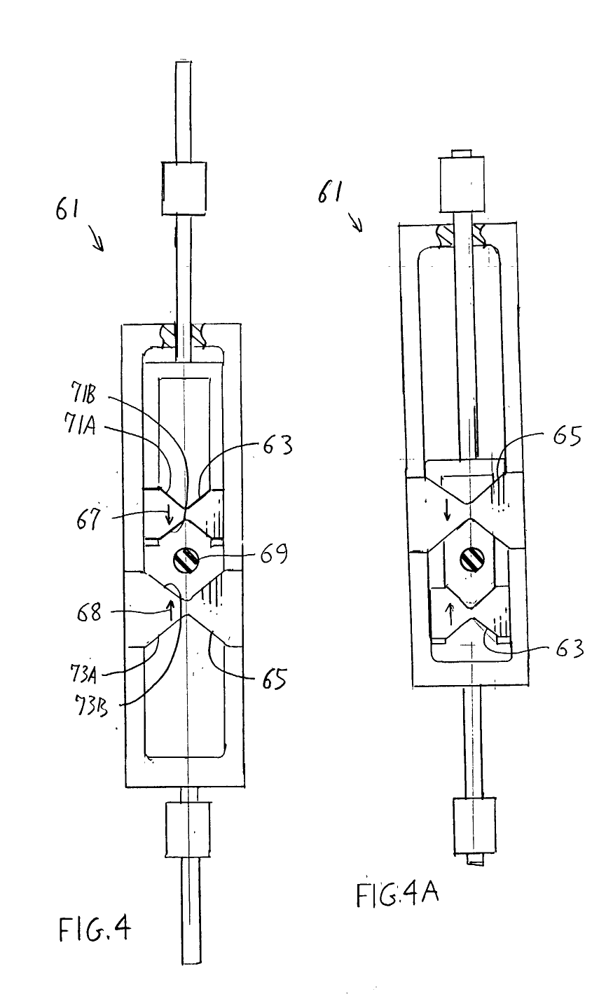

FIGS. 4 and 4A show another cutting apparatus 61 which includes a pair of blades 63, 65 that each has two cutting or shearing edges 71A, 71B, 73A, 73B. In FIG. 4, cutting is achieved by moving blade 63 downward along arrow 67 and moving blade 65 upward along arrow 68 to shear the extrusion 69 between them. The blades end in the positions of FIG. 4A. From FIG. 4A blade 63 is moved upward while blade 65 is moved downward to again shear the extrusion 69 between them. Since the blades 63, 65 do not have to stop and reverse direction, they can cut the extrusion faster and avoid having significant extrusion flowing against the blades.

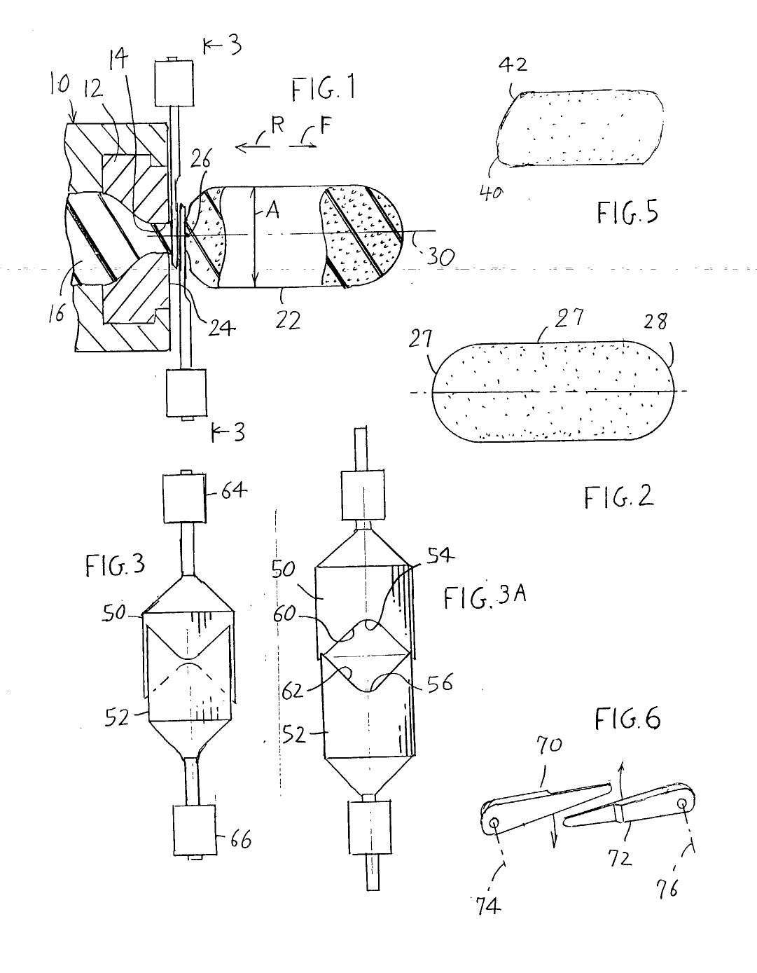

The use of a plurality of cutting, or shearing blades results in a more symmetric earplug end at the instant of severing the earplug from the material initially being extruded, resulting in the severed end growing while it foams to a more symmetric configuration. As mentioned above, an earplug end that is more symmetric about the earplug axis 30 results in an earplug that appears to be of higher quality and which is easier to insert into the ear canal. It is found that foamable material that is just beginning to foam, does not stick to steel cutting blades. The centers 54, 56 of the cutting edges shown in FIGS. 3 and 3A are preferably concavely curved. The concave edges tend to push material at opposite sides of the edge towards the axis of the earplug for greater symmetry. Three or more cutting blades can be used which move in different directions toward one another, for even greater symmetry. However, there is generally no advantage of more than six blades, but only more complexity. FIG. 6 shows a pair of cutting blades 70, 72 which rotate about corresponding axes 74, 76 in opposite directions to cut the extrusion between them, preferably with a shearing cut. Rotation of the blades 70, 72 is timed with the rate of movement of the extrusion out of the head, to produce earplugs of predetermined lengths. FIG. 2 illustrates a finished earplug 27 resulting from the processes, with rounded ends 28, 29.

The extruded material 16 in FIGS. 1 and 2 is a rapid recovery resilient foamable or foamed polymer material. A rapid recovery foam returns to its original shape within a second of moderate compression, compared to slow recovery foam that requires a plurality of seconds to return to 95% of its original diameter when moderately compressed (to 30% of its original thickness). The earplugs have an outside diameter of about 0.4 inch (10 mm). The end of the finished earplug that first centers the ear canal is referred to as the front end.

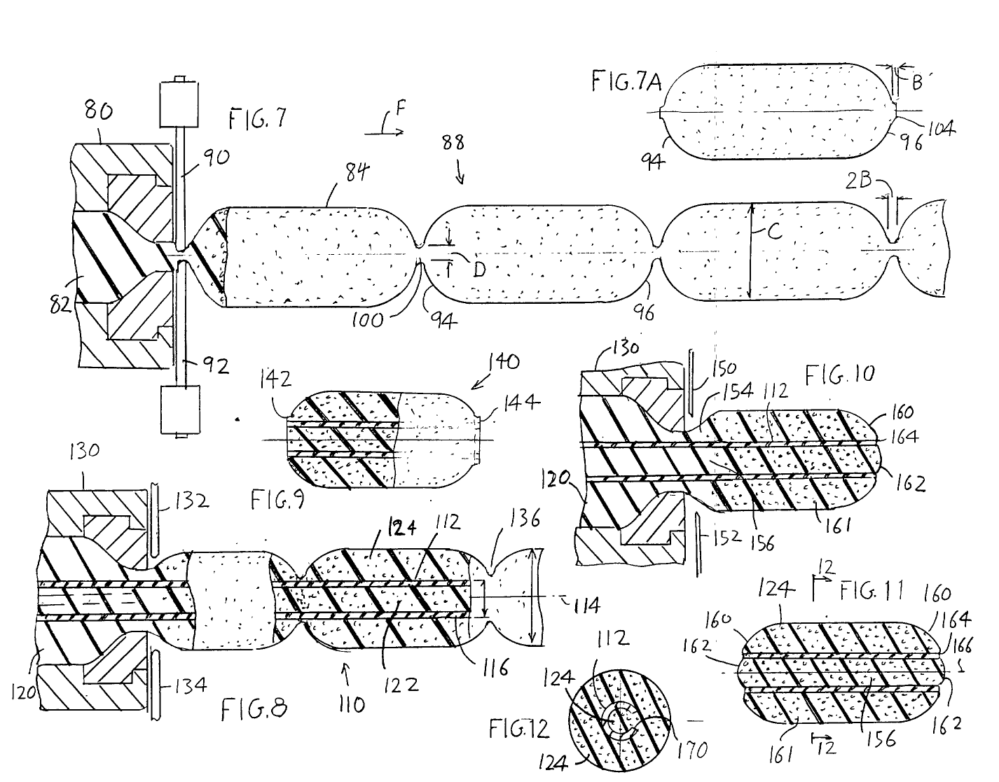

FIG. 7 illustrates the use of an extrusion head to extrude foamable material into an extrusion 84 which forms a chain of earplug elements or earplugs 88, that can be cut into individual earplugs. At intervals, a plurality of compressing dies 90, 92 compress the extrusion immediately forward F of the extrusion head. The result is that each earplug of the chain has opposite ends 94, 96 that are rounded except at a pinch location 100 where the compressing dies have pinched the foamable material. When earplugs are required by a worker, the last earplug in the chain is cut at its pinched location 100 to separate it from the rest of the chain. As shown in FIG. 7A, which shows an earplug separated from the chain, each earplug has rounded ends 94, 96, except for slight nipples or protrusions 102, 104 of lengths B. The pinched locations 100 between the rounded portions of adjacent earplugs each has a diameter much less than half the maximum diameter C of the earplug, and preferably less than one-fourth as much, so the projections 102, 104 generally will not interfere with insertion of an earplug end into the ear canal. Since the projections such 102 are formed of soft resilient foamable material, they will not hurt if scraped against the ear canal.

FIG. 8 illustrates another method for generating a chain of earplugs 110 wherein each earplug has a core 112 that resists column collapse along the axis 114. The particular core 112 is in the form of a sleeve 116 of material that is more rigid than the foamable first material 120 after it has foamed and solidified to form a resilient foam polymer material. The inside of the sleeve is filled with a string 122 of resilient material which is preferably the same as the material of the covering 124 that surrounds the core.

As the extrusion emerges from the extrusion head, a plurality of extrusion dies 132, 134 pinch it. The result is a chain of earplugs with the core 112 extending continuously through the chain. The last earplug in the chain is severed from the chain by cutting it at a pinched location 136. FIG. 9 illustrates an earplug 140 that has been cut from the chain. The ends of the earplug have large flat spots 142, 144. It is possible to compress, or pinch, the locations 136 so there is no covering 124 at the pinched locations.

The resilient foam material of the covering 124 preferably has a durometer of 1 to 10 shore A, such as 2 shore A. The material of the core has a durometer of about 30 shore A, so the core material is at least twice as stiff as the covering material. The core material is preferably a solid (no foam cells) elastomeric material such as a medium soft thermoplastic elastomer.

FIG. 10 illustrates another method for forming earplugs similar to that of FIG. 8, wherein a core 112 passes through an extrusion head 130 along with the foamable first material 120. However, instead of pinching the end of each earplug, applicant severs it by a pair of cutting blades 150,152. As a result of such cutting, the covering 154 adjacent to the extrusion head 130 expands in a bulge, as does the string 156 that fills the sleeve core 112. The final earplug shown in FIG. 11 shows that the result is convexly rounded ends 160 of the outer covering 161 and convexly rounded bulging ends 162 of the string. The sleeve ends 164 are cut perpendicular to axis 166 of the earplug.

FIG. 12 shows that the sleeve 112 is of C-shaped cross-section, with a gap 170 of about 60° through which the foamable polymer material can pass from the outside of the sleeve into the inside of the sleeve. Instead of using a sleeve-shaped core, a solid core can be used. In FIG. 11, the sleeve has an outer diameter of about 4 mm while the covering outer diameter is about 10 mm. A solid sleeve of about 2 mm diameter can be used instead.

FIG. 13 illustrates another method for forming an earplug 180. The method includes applying compression die assemblies 182,184 to form narrow bands 190,192 where the material of the earplug is radially compressed. The compression occurs before the foamed material has completely solidified. Such compression stiffens the portion of the earplug that enters the ear canal against column-type collapse, without hardening the earplug portions that engage the walls of the ear canal. The end 194 of the earplug that enters the ear canal, is cut by a pair of cutting blades 200, 202 from the extrusion, at the extrusion head 204. The end 204 of the earplug that lies outside the ear canal, is compressed by a plurality of axially wide compression dies 210, 212 to form the earplug end 204 to a high density and greater stiffness for pushing against while inserting a portion of the earplug into the ear canal.

It is noted that the foamable material can be easily shaped without hardening, and remains highly resilient, a short distance (e.g. within about one centimeter) forward (in the direction of extrusion movement) of the die. Within a moderate distance (between about one or two centimeters and a few inches) downstream of the extrusion head, the foamed material can be permanently deformed by compression, although the material then become far less resilient (i.e. stiffer) although not as stiff as the sleeve core material.

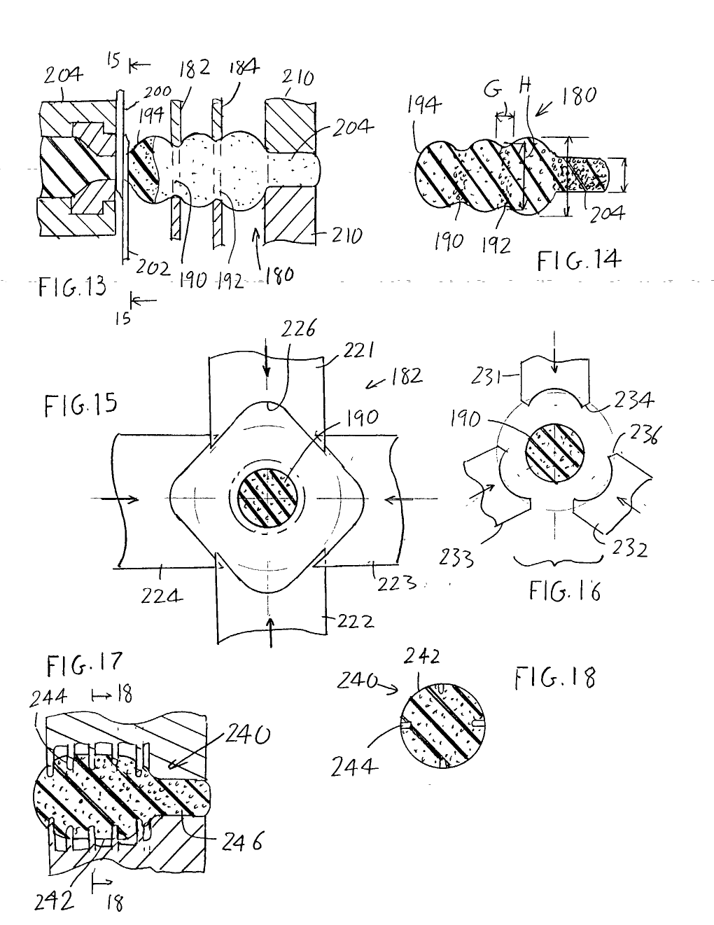

FIG. 14 shows the earplug 180 after if has been formed, with the compressed rear portion at 204 and with the compression bands at 190,192. The front end 194 of the earplug that enters the ear canal, is rounded. Each band in the earplug front portion has a band axial width G less than the band diameter H and preferably less than half, or preferably no more than one-quarter, as great as the band diameter.

FIG. 15 shows that the shaping members, or compression die assembly at 182 include four compression dies 221-224. The dies are shown as of a type that overlap one another and that have concave compression edges 226 that form the earplug band 190 so it is substantially circular.

FIG. 16 shows another compression die arrangement 230 which includes three dies 231-233 that have side surfaces such as 234, 236 that abut one another when they are moved fully together to compress a band of the earplug into the band 190. Each pair of dies such as 231, 232, or 232, 233 move primarily linearly in largely opposite directions (directions angled over 90° from each other).

FIGS. 17 and 18 show another earplug 240 whose front portion 242 that enters the ear canal is stiffened against column-type collapse by a plurality of narrow depressions 244. The rear portion 246 is more highly compressed.

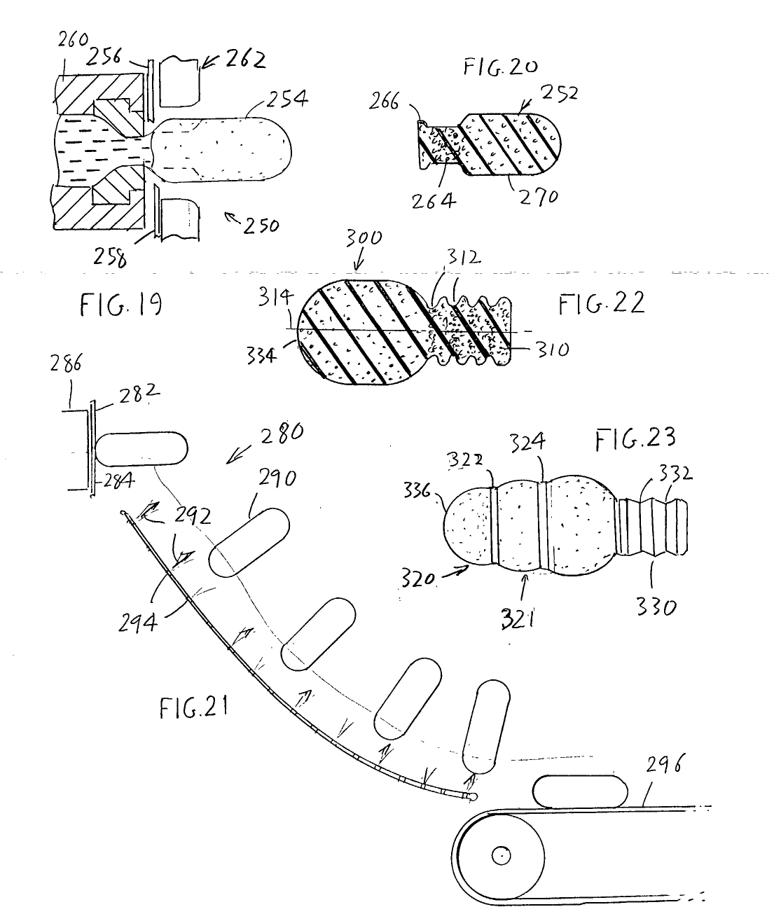

FIG. 19 shows apparatus 250 for forming an earplug of the construction shown at 252 in FIG. 20. In FIG. 19, the extrusion 254 is cut by a pair of cutting blades 256, 258 immediately in front of the extrusion head 260. The extrusion is compressed by a plurality of compression dies 262, which may include perhaps three or four compression dies of the type illustrated in FIGS. 15 and 16, to form a compressed rear end of the earplug.

FIG. 20 shows that the rear end at 264 is compressed except for the rear tip at 266. The compressed rear end 264 enables the front end 170 to be pressed into the ear canal. The compression dies 262 must be spaced sufficiently from the extrusion head to stiffen the front portion of the earplug rear end 264.

FIG. 21 illustrates an apparatus 280 for the production of earplugs by severing them with cutting blades 282, 284 at the extrusion head 286. As the severed lengths of extrusion, which form earplugs 290, fall, their rate of fall is slowed by largely upwardly directed hot air 292 passed out of directed openings 294. The upward draft of hot air is at a lower temperature than the extrusion temperature, and allows the earplugs to more gradually drop in temperature until they reach a conveyer belt 296.

FIGS. 22 and 23 illustrate earplugs 300, 320 of different shapes that can be formed by the methods of the invention. In FIG. 22, the rear end 310 of the earplug is stiffened by several compression bands 312 where the earplug is compressed radially with respect to its axis 314, to a diameter less than 75% of its maximum diameter, to stiffen the rear end against column collapse.

FIG. 23 shows an earplug 320 with a front portion 321 having compression bands 322, 324 that stiffen the front end, and with the rear end 330 compressed to a smaller diameter by bands 332 to form a rear end that is stiffer to resist collapse. The earplugs of FIGS. 22 and 23 have front ends 334, 336 that are cut from the extrusion adjacent to the extrusion head.

Thus, the invention provides an extruded earplug apparatus and methods and construction equipment. The extrusion can be severed at the extrusion head by a plurality of cutting blades that move in largely opposite directions to produce a largely symmetrical cut that allows the end of the earplug to foam and grow into a largely symmetrical rounded end that is preferred for earplugs. A chain of earplugs can be formed by compression dies that compress the extrusion at intervals to form compression locations, so when individual earplugs are cut from the end of the chain the earplugs have largely rounded ends except for small nipples where each earplug was severed from an adjacent earplug of the chain. The earplugs can be stiffened by a core that passes through the extrusion head along with foamable polymer material that forms a covering around the core. The resulting extrusion can be severed at the extrusion head to immediately form individual earplugs, or can be merely compressed at the extrusion head to form a chain of earplugs with somewhat rounded ends. The core can be in the form of a sleeve that is filled with foam polymer material, and the sleeve can have a gap in it to allow the same foamable polymer material that covers the sleeve-shape core to also fill the inside of the sleeve. Extruded earplugs can be shaped by compressing dies applied to them. Narrow compressing dies, of an axial width less than half the maximum diameter of the earplug, can be applied at locations between opposite ends of the earplug to partially radially compress the foam material to stiffen it against column-like compression. A rear end of the earplug can be compressed to a diameter no more than 60% of the maximum diameter of the earplug to form a stiffener handle. The compression dies preferably include at least two dies. Applicant can use pin-shaped dies to form small areas where the earplug is compressed to stiffen it. The fact that an earplug has been extruded can be determined by closely examining it.

Although particular embodiments of the invention have been described and illustrated herein, it is recognized that modifications and variations may readily occur to those skilled in the art, and consequently, it is intended that the claims be interpreted to cover such modifications and equivalents.

What is claimed is:

1. An earplug apparatus comprising:
an earplug comprising resilient foam polymer material that has been extruded through an extrusion opening, said earplug having an axis, and said earplug having an outer surface that is partly cylindrical;
said earplug outer surface includes at least one region centered on said axis where said polymer material has been radially inwardly compressed from at least two largely opposite sides.

2. The apparatus described in claim 1 wherein:
said earplug has opposite ends spaced along said axis, and said at least one region includes a plurality of band shaped regions lying between said ends, each of said band shaped regions having a length along said axis which is no more than half the diameter of the band-shaped region.

3. The apparatus described in claim 1 including:
a rear band shaped region forming a handle and having a cross-section taken perpendicular to the axis of no more than about 60% of the maximum cross-section of the earplug.

4. The apparatus described in claim 1 wherein:
said earplug has locations of maximum diameter, and said earplug has a front portion that enters the ear canal and a rear portion that serves as a handle;
said plurality of band-shaped regions include a band along said front portion, where the outside diameter of the earplug has been reduced to between 60% and 90% of the maximum diameter of the earplug.

5. The apparatus described in claim 1 including:
a chain of earplug elements, each having a length between about 10 to 40 millimeters, with sequential earplug elements along the chain being partially separated by a separation location which has at least about a 50% reduction in diameter so an earplug element at the end of the chain can be separated from the rest of the chain by cutting a separation location;
said earplug being one of said earplug elements of said chain.

6. The apparatus described in claim 5 wherein:
said chain of earplug elements has a maximum outside diameter and includes a stiffening core having a core outside diameter no more than half of said maximum outside diameter, and each of said earplug elements includes said foam polymer material surrounding said core;
at said separation locations, the thickness of polymer material surrounding said core is less than about one-quarter the average thickness of said polymer material along the length of the earplug elements.

7. An extruded earplug apparatus, comprising:
an earplug which has a substantially cylindrical stiffening sleeve with an axis, a covering surrounding said sleeve and a string lying within and filling said sleeve, said covering and said string each constructed of a resilient foam polymer first material and said sleeve having sleeve walls of a second material that is stiffer than said first material;
said earplug having a first end, and at said first end of said earplug said sleeve has an end that is cut substantially perpendicular to said axis, said covering has an end that is convexly rounded, and said string has an end that is convexly rounded.

8. The apparatus described in claim 7 wherein:
said string has opposite ends that are each convexly rounded.

9. The apparatus described in claim 7 including:
a plurality of earplug devices extending in a chain, said earplug being one of said earplug devices;
said sleeve and string extending continuously along said chain and having constant diameters along said chain;
said covering having a reduced outside diameter at the opposite ends of each earplug device to define the earplug devices along the chain.

10. An earplug apparatus, comprising:
an earplug of resilient polymer foam material, said earplug having an axis and having locations of a maximum outer diameter;
said earplug has at least one radially inwardly deformed band of said polymer material where said resilient polymer foam material is stiffer than at said locations of maximum outer diameter.

11. The apparatus described in claim 10 wherein:
said earplug has opposite ends and said at least one band includes a plurality of bands of radially inward deformation spaced from said opposite ends, where said resilient polymer foam material is stiffer than at said locations of maximum outer diameter.

12. An earplug apparatus, comprising:
a chain of earplugs that has an axis and that includes a core and a covering that covers the core, the core being constructed of material at least twice as stiff as the material of the covering;
said core having a constant outer diameter along the chain and said covering having a maximum thickness along said chain, but said covering having locations spaced by about 10 mm to 50 mm along the axis to define the beginning and end of each earplug and where the covering thickness is less than half the covering thickness at locations of said maximum thickness.

13. The apparatus described in claim 12 wherein:
said core is in the form of a sleeve.

14. The apparatus described in claim 13 wherein:
said sleeve has a slot extending along its length and said sleeve is filled with a string of the same material as said covering.

15. Apparatus for use in forming earplugs by extruding foamable material through an extrusion die, by also cutting the extrusion into pieces as the extrusion emerges from the extrusion die, comprising:
a pair of cutting blades and at least one actuator for moving the blades in opposite directions across the extrusion to cut the extrusion;
each of said cutting blades has two cutting edges, and said actuator moves said blades in first and second directions completely across said extrusion to cut the extrusion a first time with first blade edges, and then in second and first directions to cut the extrusion a second time with second blade edges.

16. A method for constructing earplugs which includes extruding polymer material which is foamable through an extrusion head opening, to form an extrusion with an axis, and allowing the extruded polymer material to foam and expand in diameter and solidify, comprising:
applying compression forces from at least two largely opposite radial directions to the extrusion prior to complete solidification of the polymer material, to permanently deform the polymer material in compression.

17. The method described in claim 16 wherein:
said step of applying compression forces includes moving a plurality of shaping members that each has a concave compression edge, toward said axis from largely opposite sides of each axis.

18. A method for constructing earplugs which includes extruding polymer material which is foamable, through an extrusion head opening to form an extrusion with an axis, and allowing the extruded polymer material to foam and expand in diameter and solidify, which includes severing the extrusion near the extrusion head to separate the extrusion into individual earplugs while allowing the ends of the earplugs to expand and solidify, comprising:
moving a plurality of cutting blades into largely opposite sides of the extrusion at a location adjacent to the extrusion head, until a portion of the extrusion of more than 10 mm length that lies forward of the head is severed from material that is emerging from the head, to produce a largely symmetrical cut end of the extruded polymer material that allows the cut end to grow into a largely symmetrical rounded end.

19. The method described in claim 18 wherein:
said extrusion head opening has an axis and said step of moving a plurality of cutting blades includes rapidly sliding cutting edges of said blades closely adjacent to one another and toward and substantially beyond said axis to shear the extrusion between them.

20. The method described in claim 18 wherein:
said step of moving a plurality of cutting blades includes rotating a pair of cutting blades about spaced axes so their cutting edges move across one another in largely opposite directions and simultaneously across the extrusion.
